(19) United States
(12) Patent Application Publication
(10) Pub. No.: US 2017/0049389 A1
(43) Pub. Date: Feb. 23, 2017

(54) Internet-Based Traditional Chinese Medical Science Health Consultation System

(71) Applicant: Zhiyi (Shanghai) Health Technology, Shanghai (CN)

(72) Inventors: Xiaoyan Gu, Shanghai (CN); Rui Ren, Shanghai (CN); Zhengli Zhang, Shanghai (CN); Tangyi Liu, Shanghai (CN); Daopeng Jin, Shanghai (CN); Yuan Qin, Shanghai (CN); Haoran Sun, Shanghai (CN)

(21) Appl. No.: 15/233,871

(22) Filed: Aug. 10, 2016

(30) Foreign Application Priority Data
Aug. 19, 2015 (CN) 201510510624.2

(51) Int. Cl. A61B 5/00; A61B 5/024; H04N 7/14; G06F 19/00

(52) U.S. Cl. CPC A61B 5/4854; G06F 19/3418; G06F 19/363; A61B 5/02416; H04N 7/147

(57) ABSTRACT

The invention discloses a traditional Chinese medicine health consultation system based on the internet. The traditional Chinese medicine health consultation system improves the traditional Chinese medicine health consultation efficiency. According to the technical solution, the system comprises a client side and a server. Face and tongue picture taking, sound recording, questionnaire surveying and radial artery pulse picture taking are carried out by the client side on a subject. The collected information is processed and then sent to the server, the information is intelligently processed based on a preset data model in the server, an obtained automatic health consultation result is fed back to the subject, the server can also forward the information collected by the client side to a client side for a doctor, and the doctor makes a health consultation result according to the information and provide feedback of the health consultation result to the subject.

### CROSS-REFERENCE TO RELATED APPLICATION

This application claims the benefit of China Application No. 201510510624.2, filed Aug. 19, 2015, which is hereby incorporated by reference in its entirety.

### BACKGROUND OF THE INVENTION

1. Field of the Invention

The invention relates to a type of traditional Chinese medical science health consultation system. More specifically the invention relates to an internet based and mobile internet based remote traditional Chinese medical science health consultation system.

2. Background

Traditional Chinese medical science health consultation requires a doctor and a subject to communicate face to face. A subject has to go to a hospital or a clinic in person to register, line up and talk to a doctor about the sickness, and the doctor also needs to timely collect the subject's health information and pulse diagnosis information, and do synthetic analysis to understand the subject's health status.

In order to check a subject's health status, traditional Chinese medical science must use four diagnosis methods: observing, listening, asking and pulse feeling. Observing is watching the subject's mental condition, face color, figure and tongue etc. Listening is checking the subject's voice, e.g. speaking or coughing. Asking is inquiring the subject about any information related to the subject's health status. Pulse feeling is for pulse diagnosis. Traditional Chinese medical science believes that a form of pulse condition is closely related to viscera's qi and blood status, if viscera's qi and blood status has changed because of illness, blood flow will be impacted and then pulse condition will change. In the clinic, pulse diagnosis is used for inferring a place and a property for a sick organ and also sickness' progressing, fading-away and prognosis.

Traditional Chinese medical science's four diagnosis methods: observing, listening, asking and pulse feeling requires face to face communication between a doctor and a subject, which is obviously very time consuming, and it is difficult to improve efficiency of pulse diagnosis in traditional Chinese medical science.

Now, with an internet era, the Chinese government brings up the internet and the concept, and transforms the traditional industry to an internet based industry, which will improve working efficiency in the traditional industry.

Therefore, based on the internet, how to improve efficiency in traditional Chinese medical science health consultation industry is one urgent problem to be solved in this industry.

### SUMMARY

A summary of one or multiple aspects below will provide a basic understanding of those aspects. The summary is not a detailed elaboration of all aspects and it is neither going to recognize critical or crucial facts for all aspects nor is it going to create a scope for any or all aspects. The purpose is to provide some concept for one or more aspects in order for a more detailed description.

The purpose of this present invention is to solve the above problems by providing an internet-based traditional Chinese medical science health consultation system. Based on the internet, remote traditional Chinese medical science health consultation is developed to greatly improve the efficiency of traditional Chinese medical science health consultation. The internet reduces time and distance for traditional Chinese medical science health consultation. Moreover, it uses a predefined model in the server to perform intelligent health consultation and realizes high efficiency, popularity and intelligence for traditional Chinese medical science health consultation.

The technical solution for this invention is that it discloses an internet-based traditional Chinese medical science health consultation system, including a first client used for a subject to login for traditional Chinese medical science health consultation, including an image acquiring module, the image acquiring module acquires the subject's image for traditional Chinese medical science health consultation. A voice inspection module, acquires the subject's voice for traditional Chinese medical science health consultation. A questionnaire investigation module, acquires a subject's questionnaire information for traditional Chinese medical science health consultation. A pulse diagnosis module, acquires the subject's pulse condition information for traditional Chinese medical science health consultation.

A first network communication module, an image acquiring module, a voice inspection module, a questionnaire investigation module, and a pulse diagnosis module, encapsulates image, voice, questionnaire and pulse condition information and then transmits them to a server.

A second client is used for a doctor to login, which includes a second network communication module, which is used to communicate with a first client and a server. A second client receives data collected from a first client and sends the health consultation feedback from a doctor to a first client.

A health data visualization module is used for receiving an image and a voice transmitted from a server and displaying them to a doctor.

A server, includes a server interface module that converts data from a client side to an image, a voice, a questionnaire information and pulse condition information.

The storage module, stores data from the client side.

According to an embodiment of the internet-based traditional Chinese medical science health consultation system of the present invention, the server also includes a data processing module, which processes an image, a voice, a questionnaire information and a pulse information based on which intelligent health consultation is conducted. The intelligent health consultation module, based on the predefined data analytics model and the output from the data processing module, provides an automatic health consultation result, and then transmits the result to the first client via the server interface module.

According to another embodiment of the internet-based traditional Chinese medical science health consultation system of the present invention, the first client includes the first network communication module, which is used for communicating with the second client; the second client includes a second network communication module, which is used for communicating with the first client.

According to an embodiment of the internet-based traditional Chinese medical science health consultation system of the present invention, the data processing module includes an image processing unit, the image processing unit preprocesses the image, which the subject uploads and normalizes the luminance and the color of this image.

The voice processing unit, stores voice which the subject uploads to the sound file. The questionnaire information processing unit, stores the questionnaire, scores the questionnaire and stores the score results.

The pulse information processing unit, based on a predefined ANN (Artificial Neural Network) model, processes pulse information, performs intelligent health consultation and provides pulse diagnosis result. According to one embodiment of the internet-based traditional Chinese medical science health consultation system of the present invention, the pulse diagnosis module includes a pulse image collecting unit, which collects the pulse image from the subject. The pulse image preprocessing unit, is connected to the pulse image collecting unit, the pulse image preprocessing unit does preprocessing of pulse image collected from the client. The pulse condition extraction unit, is connected to the pulse image collecting unit, the pulse condition extraction unit extracts the pulse condition information from the data after preprocessing.

According to an embodiment of the internet-based traditional Chinese medical science health consultation system of the present invention, the pulse image preprocessing module includes an image sampling unit, which extracts samples out of the pulse images in a fixed frequency and gets multiple frames of the pulse images.

The image clipping unit is connected to the image sampling unit. It finds out the area with the highest luminance for each of the frame of the pulse image, and uses a uniform rectangle window to clip. The noise repairing unit, is connected to the image clipping unit. The noise repairing unit repairs the noise points in the window after clipping.

According to another embodiment of the internet-based traditional Chinese medical science health consultation system of the present invention, a pulse condition extraction module includes three primary colors, the average computing unit, which calculates the average value of the three primary colors for each frame of the pulse image.

A red primary color variation range computing unit is based on the average red primary color in the average three primary colors for each frame of the pulse image, and calculates the variation range for a red primary color. The pulse frequency computing unit, which is based on the red primary color variation range for each frame of the pulse image, calculates pulse frequency.

According to an embodiment of the internet-based traditional Chinese medical science health consultation system of the present invention, a pulse condition includes the average of three primary colors for each frame of the pulse image, the variation range for red primary color and pulse frequency.

According to an embodiment of the internet-based traditional Chinese medical science health consultation system of the present invention, an image acquiring module acquires the subject's face and tongue image.

According to an embodiment of the internet-based traditional Chinese medical science health consultation system of the present invention, a first client and a second client are at the mobile terminal.

An embodiment of this invention also reveals one internet-based traditional Chinese medical science health consultation system, including a client used by a subject to login for traditional Chinese medical science health consultation, including an image acquiring module, which acquires the subject's image for traditional Chinese medical science health consultation.

A voice inspection module, acquires a subject's voice for traditional Chinese medical science health consultation.

A questionnaire investigation module, acquires a subject's questionnaire information for traditional Chinese medical science health consultation. A pulse diagnosis module, acquires a subject's pulse condition information for traditional Chinese medical science health consultation.

A first network communication module, an image acquiring module, a voice inspection module, a questionnaire investigation module, and a pulse diagnosis module, which encapsulates the image, the voice, the questionnaire and the pulse condition information and then transmits them to a server.

A server, includes a server interface module, which converts the data from the client side to image, voice, questionnaire information and pulse condition information. A storage module, stores data from the client side. A data processing module, which processes an image, a voice, a questionnaire information and a pulse information based on which intelligent health consultation is conducted.

The intelligent health consultation module, which is based on predefined data from the analytics model and the output from the data processing module, provides an automatic health consultation result, and then transmits the result to a first client via a server interface module.

According to an embodiment of the internet-based traditional Chinese medical science health consultation system in this invention, the data processing module includes: an image processing unit, which does the preprocessing of image which the subject uploads and normalizes the luminance and color of this image.

The voice processing unit, stores voice which the subject uploads to the sound file. The questionnaire information processing unit, stores the questionnaire, scores the questionnaire and then stores score result.

The pulse information processing unit, which is based on predefined ANN (Artificial Neural Network) model, processes the pulse information, performs intelligent health consultation and provides the pulse diagnosis result.

According to an embodiment of the internet-based traditional Chinese medical science health consultation system in this invention, the pulse diagnosis module includes: the pulse image collecting unit, which collects the pulse image from the subject.

The pulse image preprocessing unit, is connected to the pulse image collecting unit, which performs preprocessing of the pulse image collected from the client. The pulse condition extraction unit, is connected to the pulse image collecting unit, which extracts the pulse condition information from the data after preprocessing.

According to an embodiment of the internet-based traditional Chinese medical science health consultation system in this invention, the pulse image preprocessing module includes: the image sampling unit, which extracts the samples out of the pulse images in a fixed frequency and gets multiple frames of pulse images.

The image clipping unit, is connected to the image sampling unit. It finds out the area with the highest luminance for each frame of the pulse image, and uses the uniform rectangle window to clip.

The noise repairing unit, is connected to the image clipping unit. The noise repairing unit repairs noise points in the window after clipping.

According to an embodiment of the internet-based traditional Chinese medical science health consultation system in this embodiment of the present invention, a pulse condition extraction module includes: three primary colors, an average computing unit, which calculates the average value of three primary colors for each frame of the pulse image.

A red primary color variation range computing unit, based on an average red primary color in average three primary colors for each frame of pulse image, calculates the variation range for the red primary color.

The pulse frequency computing unit, based on red primary color variation range for each frame of a pulse image, calculates the pulse frequency.

According to an embodiment of the internet-based traditional Chinese medical science health consultation system in the embodiment of the invention, the pulse condition includes an average of three primary colors for each frame of the pulse image, the variation range for the red primary color and the pulse frequency.

According to an embodiment of the internet-based traditional Chinese medical science health consultation system in this invention, Image Acquiring Module acquires a subject's face and tongue image.

Compared with existing technology, this embodiment of the present invention has the following good benefits: the system includes a client and a server. A client is installed as mobile APP in a smart phone. A client takes photos of the subject's face and tongue, records the subject's voice, provides a questionnaire, takes video shooting of the subject's pulse, preprocesses the video, extracts a pulse condition and sends to the server a photo of the face and tongue, the recorded voice, the questionnaire information and the pulse condition. In the end, the server conducts an intelligent health consultation based on a predefined data model and transmits the health consultation result to the subject's client. The server can also transmit those information collected to a doctor side's smart phone terminal and the doctor can determine a health consultation result and transmit it to the subject's smart phone terminal. Moreover, a doctor and a subject can have interactive communication through a smart phone terminal, which further improves the effects of the health consultation.

### BRIEF DESCRIPTION OF THE DRAWINGS

In order to more fully describe embodiments of the present invention, reference is made to the accompanying drawings. These drawings are not to be considered limitations in the scope of the invention, but are merely illustrative.

FIG. 1 shows a schematic for Embodiment 1 of this invention's internet-based traditional Chinese medical science health consultation system.

FIG. 2 shows a schematic for Embodiment 2 of this invention's internet-based traditional Chinese medical science health consultation system.

FIG. 3 shows a schematic for Embodiment 3 of this invention's internet-based traditional Chinese medical science health consultation system.

FIG. 4 shows a schematic for Embodiment 4 of this invention's internet-based traditional Chinese medical science health consultation system.

FIG. 5 shows a detailed schematic of the Pulse Diagnosis Module, according to an embodiment of the present invention.

### DETAILED DESCRIPTION OF SPECIFIC EMBODIMENTS

The description above and below and the drawings of the present document focus on one or more currently preferred embodiments and also describe some exemplary optional features and/or alternative embodiments. The description and drawings are for the purpose of illustration and not limitation. Those of ordinary skill in the art would recognize variations, modifications, and alternatives. Such variations, modifications, and alternatives are also within the scope of the present invention. Section titles are terse and are for convenience only.

By reading the description of this published embodiment together with the figures, people can better understand the above characteristics and advantages of the present invention. Every component is not necessarily drawn according to its correct proportion and those components with similar characteristics may have the same or similar marks in the figures.

A First Embodiment of the Internet-Based Traditional Chinese Medical Science Health Consultation System.

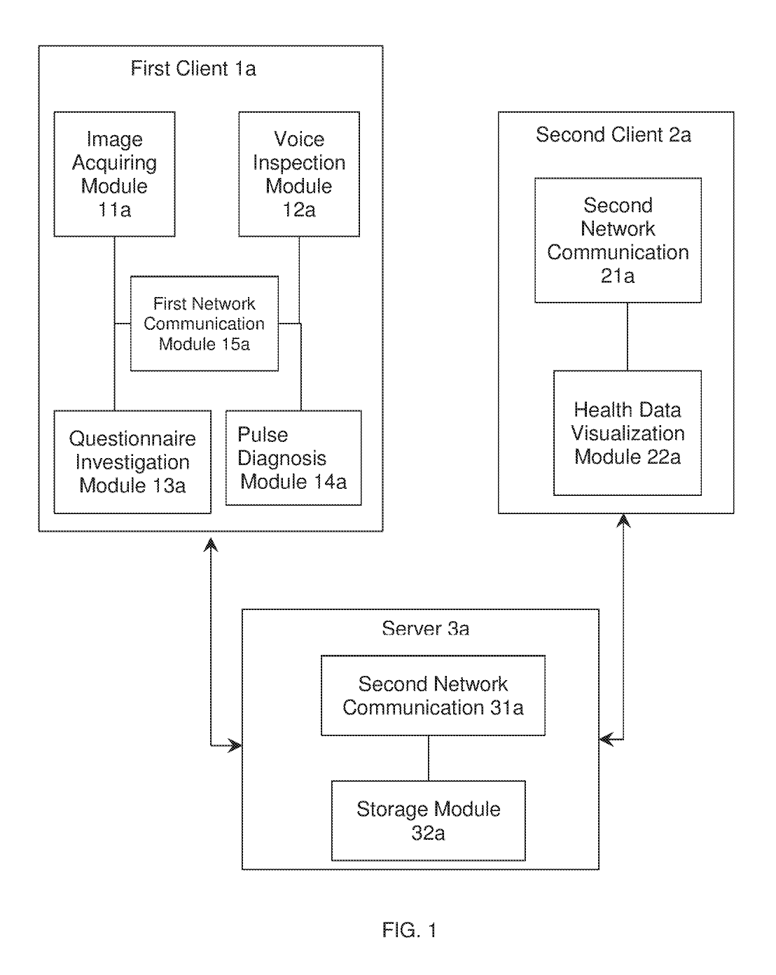

FIG. 1 shows principles for Embodiment 1 of the internet-based traditional Chinese medical science health consultation system. In reference to FIG. 1, this embodiment for the internet-based traditional Chinese medical science health consultation includes a client 1a for a subject's login, a client 2a for the doctor's login and a Server 3a. The client 1a and the client 2a are good to be at a mobile terminal, for example they can be an APP installed in a smart phone. When logging in as a subject, this smart phone becomes Client 1a. When logging in as a doctor, this smart phone becomes Client 2a.

The client 1a includes the image acquiring module 11a, the voice inspection module 12a, the questionnaire investigation module 13a, the pulse diagnosis module 14a and the first network communication module 15a. The first network communication module 15a connects electrically with the image acquiring module 11a, the voice inspection module 12a, the questionnaire investigation module 13a, and the pulse diagnosis module 14a.

The image acquiring module 11a acquires an image of the subject for traditional Chinese medical science health consultation. For example in this embodiment, it takes photos of the subject's face and tongue through a smart phone's camera.

The voice inspection module 12a acquires the voice of a subject for traditional Chinese medical science health consultation. For example in this embodiment, it collects the subject's voice, e.g. reading one paragraph or cough etc.

The questionnaire investigation module 13a acquires the subject's questionnaire investigation information for traditional Chinese medical science health consultation. Its purpose is to collect the health status for the subject through questionnaire investigation.

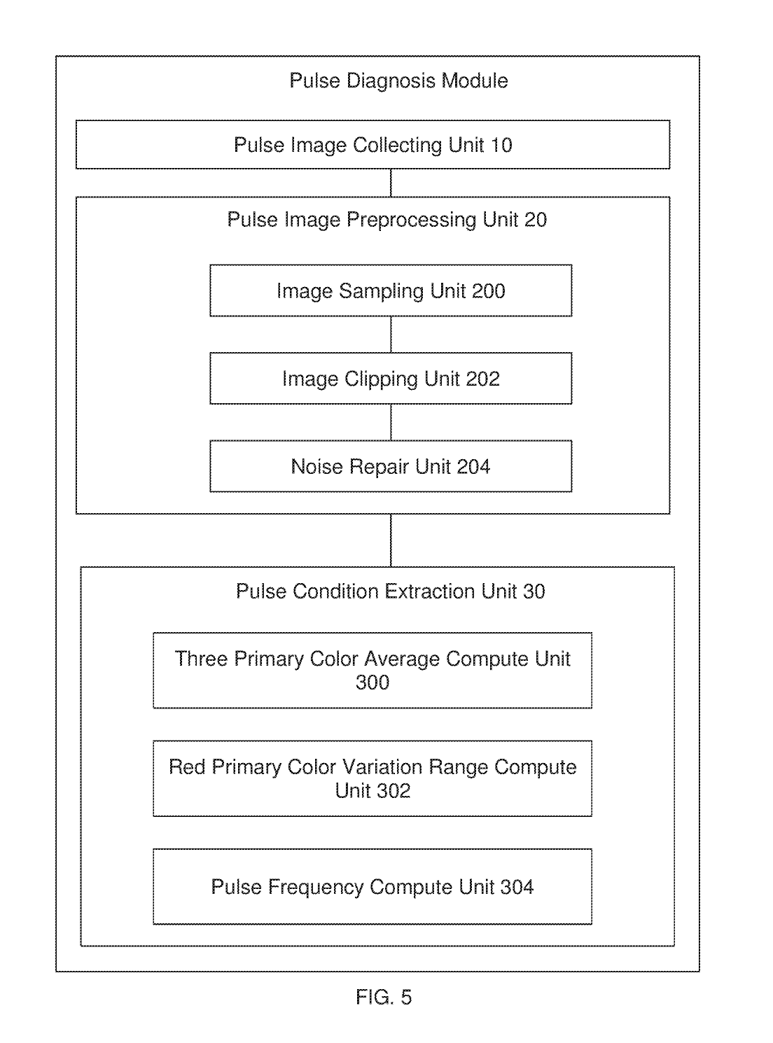

The pulse diagnosis module 14a acquires the subject's pulse condition for traditional Chinese medical science health consultation. In reference to FIG. 5, the pulse diagnosis module 14a also includes the pulse image collecting unit 10, pulse image preprocessing unit 20 and pulse condition extraction unit 30. The output of the pulse image collecting unit 10 is connected to the pulse image preprocessing unit 20. The output from the pulse image preprocessing unit 20 is connected to the pulse condition extraction unit 30. The output from the pulse condition extraction unit 30 is connected to the first network communication 15a.

The pulse image collecting unit 10 is used for collecting the subject's pulse image. For a smart phone, the pulse image collecting unit 10 is the camera components in a cell phone, including the camera, the flash etc. The subject uses the camera to do video shooting on the pulse place and may use flash on demand to make a better video effect. Usually collecting 10 seconds' video can meet the requirement.

The pulse image preprocessing 20 is used for processing collected pulse images. Further, the pulse image preprocessing unit 20 includes the image sampling unit 200, the image clipping unit 202 and the noise repair unit 204. The connection relationships among those units are: the output of the image sampling unit 200 is connected to the image clipping unit 202 and the output of the image clipping unit 202 is connected to the noise repair unit 204.

The image sampling unit 200 takes samples using fixed frequency from pulse images collected and gets multiple pulse images. The image clipping unit 202 finds out the area with the highest luminance for each frame of a pulse image, and uses a uniform rectangle window to clip. The noise repairing unit 204 repairs the noise points in the rectangular window after clipping.

The pulse condition extraction unit 30 extracts the pulse condition from preprocessed data. The pulse condition includes an average of three primary colors (R,G,B) for each frame of a pulse image, a variation range for a red primary color (R) and a pulse frequency. In traditional Chinese medical science, pulse condition refers to outside symptom from pulse beat, which includes pulse frequency, pulse rhythm, pulse position, pulse veins, pulse figure, pulse energy and pulse width. The variation of pulse condition is closely related to viscera's qi and blood function and status, therefore pulse diagnosis can provide reference data for traditional Chinese medical science health consultation.

A pulse condition extraction unit 30 also includes: a three primary colors average compute unit 300, a red primary color variation range compute unit 302 and a pulse frequency compute unit 304.

The three primary colors average compute unit 300 computes the average value for each pulse image's three primary color (R, G, B).

The red primary color variation range compute unit 302 computes variation range for the red primary color based on red primary color average value in three primary color average value for each frame of pulse image.

The pulse frequency compute unit 304 computes pulse frequency based on red primary color (R) variation range in each frame of the pulse image.

The first network communication module 15a encapsulates the above mentioned image, voice, questionnaire information and pulse condition into transmittable data and sends the data to a server 3a.

A server 3a includes a server interface module 31a and a storage module 32a. The server interface module 31a converts the collected transmittable data into an image, a voice, a questionnaire and a pulse condition, and stores them through a storage module 32a.

A second client 2a includes a second network communication module 21a and a health data visualization module 22a. A second network communication module 21a receives an image and a voice data transmitted from the server 3a, and displays those images and voice through the health data visualization module 22a.

The doctor determines the health consultation result based on visualized data and via the server 3a gives feedback to the subject's first client 1a.

A Second Embodiment of the Internet-Based Traditional Chinese Medical Science Health Consultation.

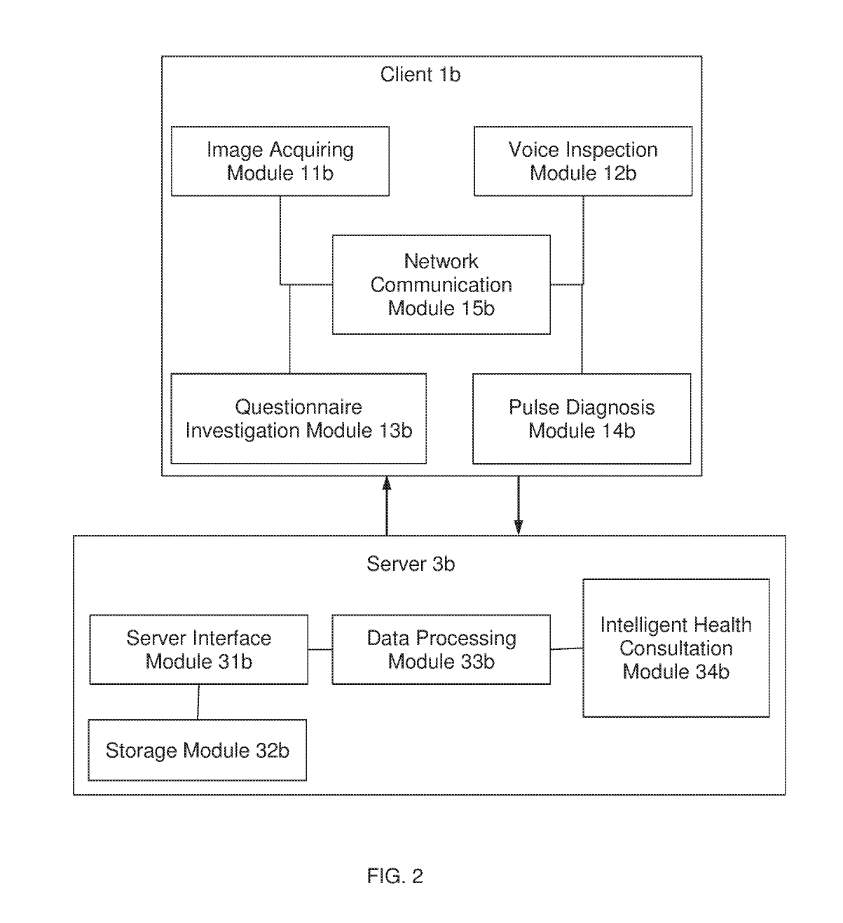

For the second embodiment of the internet-based traditional Chinese health consultation system, please reference FIG. 2. As illustrated in FIG. 2, the internet-based traditional Chinese health consultation system in this embodiment includes the client 1b for the subject to login and the server 3b. The client 1b is preferably a mobile terminal, and can be an APP installed in smart phone.

The client 1b includes an image acquiring module 11b, a voice inspection module 12b, a questionnaire investigation module 13b, a pulse diagnosis module 14b and a network communication module 15b. The network communication module 15b is electrically connected to the image acquiring module 11b, the voice inspection module 12b, the questionnaire investigation module 13b, and the pulse diagnosis module 14b respectively.

The image acquiring module 11b acquires the subject's images for traditional Chinese medical science health consultation, for example in this embodiment it takes photos of the subject's face and tongue through a smart phone's camera.

The voice inspection module 12b acquires the subject's voice for traditional Chinese medical science health consultation, for example in this embodiment it collects the subject's voice, e.g. reading one paragraph or cough etc.

The questionnaire investigation module 13b acquires the subject's questionnaire investigation information for traditional Chinese medical science health consultation. Its purpose is to collect health status for the subject through questionnaire investigation.

The pulse diagnosis module 14b acquires the subject's pulse condition. In reference to FIG. 5, the pulse diagnosis module 14b, which also includes the pulse image collecting unit 10, the pulse image preprocessing unit 20, and the pulse condition extraction unit 30. The output from the pulse image collecting unit 10 is connected to the pulse image preprocessing unit 20. Output from the pulse preprocessing unit 20 is connected to the pulse condition extraction unit 30. The output from the pulse condition extraction unit 30 is connected to the network communication module 15b.

The pulse image collecting unit 10 is used for collecting the subject's pulse image. For smart phones, the pulse image collecting unit 10 is a camera component in a cell phone, including the camera, flash etc. The subject uses the camera to do video shooting on the pulse place and may open flash on demand to make better video effects. Usually collecting 10 seconds' video can meet the requirement.

The pulse image preprocessing 20 is used for processing a pulse image collected. Further, the pulse image preprocessing unit 20 includes the image sampling unit 200, the image clipping unit 202 and the noise repair unit 204. The connection relationships among those units are: the output of the image sampling unit 200 is connected to the image clipping unit 202 and the output of the image clipping unit 202 is connected to the noise repair unit 204.

The image sampling unit 200 takes samples using fixed frequency from pulse images collected and gets multiple pulse images. The image clipping unit 202 finds out the area with highest luminance for each frame of pulse image, and uses uniform rectangle window to clip. The noise repairing unit 204 repairs noise points in rectangular window after clipping.

The pulse condition extraction unit 30 extracts the pulse condition from preprocessed data. The pulse condition includes an average of three primary colors (R,G,B) for each frame of pulse image, variation range for red primary color (R) and a pulse frequency. In traditional Chinese medical science, pulse condition refers to outside symptom from pulse beat, which includes pulse frequency, pulse rhythm, pulse position, pulse veins, pulse figure, pulse energy and pulse width. The variation of pulse condition is closely related to viscera's qi and blood function and status, therefore pulse diagnosis can provide reference data for traditional Chinese medical science health consultation.

The pulse condition extraction unit 30 also includes a three primary colors average compute unit 300, red primary color variation range compute unit 302 and pulse frequency compute unit 304. The three primary colors average compute unit 300 computes the average value for each pulse image's three primary color (R, G, B).

The red primary color variation range compute unit 302 computes variation range for red primary color (R) based on red primary color average value in three primary color average value for each frame of a pulse image.

The pulse frequency compute unit 304 computes the pulse frequency based on the red primary color (R) variation range in each frame of the pulse image.

The first network communication module 15a encapsulates above mentioned image, voice, questionnaire information and pulse condition into a transmittable data and sends the data to a server 3b.

The server 3b includes a server interface module 31b, a storage module 32b, a data processing module 33b, and an intelligent health consultation module 34b. The server interface module 31b converts the data received into an image, voice, questionnaire information and pulse condition and stores them through a storage module 32b.

The data processing module 33b processes receive an image, voice, questionnaire information and pulse condition in order to provide information for intelligent health consultation. The data processing module 33b includes an image processing unit 331b, a voice processing unit 332b, a questionnaire processing unit 333b, and a pulse condition processing unit 334b.

The image processing unit 331b preprocesses uploaded image from the subject, mainly normalizing luminance and color for face and tongue images.

The voice processing unit 332b stores uploaded voice from the subject (voice includes talking and cough) as voice file.

The questionnaire processing unit 333b stores questionnaire information and scores the questionnaire and stores the score result.

The pulse condition processing unit 334b conducts intelligent pulse diagnosis based on the pulse condition using predefined ANN (Artificial Neural Network) model and then achieves the pulse diagnosis result. For this embodiment, the pulse diagnosis result is the detailed pulse condition, for example Yin Pulse, Yang Pulse, Concurrent Pulse and Flat Pulse etc. In traditional Chinese medical science, pulse condition is firstly classified into Yin and Yang. For example in the book, Typhoid Theory. Pulse Recognizing Method, it notes: “If the pulse is big, floating, counting, moving, sliding, it is Yang; if the pulse is falling, obscure, weak, single, tiny, it is Yin.” In other words, the pulse condition is a Yan pulse if it is large in pulse figure and range, powerful, fast in pulse frequency and shallow in pulse position; and when the contrary it is a Yin pulse. Classification for the Yin and Yang pulse and its effect is consistent with the Yin and Yang proof in Eight Proofs, i.e. the Yin Proof and Yang Proof are the leading proofs in Eight Proofs. A Yin and Yang pulse dominates all pulses, and which is the basis for judging sickness exterior/interior, abstract/concrete, cold/hot. Concurrent pulse refers to a pulse with both characteristics from a Yin and Yang pulse, for example a falling counting pulse, floating slow pulse. Flat pulse refers to a pulse which comes softly, neither fast nor slow, and indicates a healthy pulse condition.

In this embodiment, the artificial neural network model is a four-layer model including three input values (three parameters in pulse condition) and one output value (pulse diagnosis result). This model is generated as follows: use a random parameter to generate it, conduct supervised learning through knowledge from an expert system module (collected and sorted out in beginning experiment stage), when the model parameter is converging, it indicates a learning step is completed and it forms an Artificial Neural Network model which can be used for health consultation based on pulse condition.

The intelligent health consultation module 34b provides an automatic health consultation result based on an output from a predefined data analytics model and data processing module 33b and then transmits that to Client 1b through Server Interface Module 31b. This data analytics model is designed based on big data technology and it is achieved by analyzing and studying historical data.

A Third Embodiment of the Internet-Based Traditional Chinese Medical Science Health Consultation.

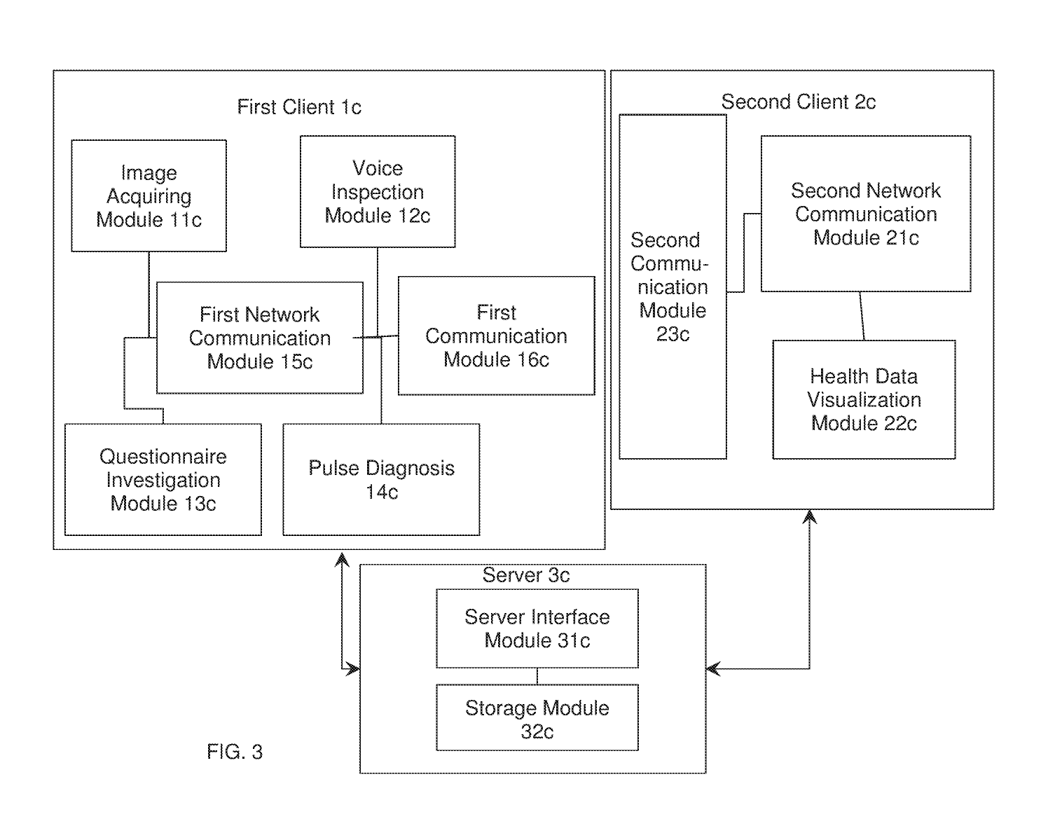

FIG. 3 shows the principle for the third embodiment of the internet-based traditional Chinese medical science health consultation. In reference to FIG. 3, this embodiment of the internet-based traditional Chinese medical science health consultation includes First Client 1c allowing the subject to login, Second Client 2c for the doctor to login, and Server 3c. Among them, First Client 1c and Second Client 2c are preferably mobile terminals, so they can be an APP installed in a smart phone. When a subject logs in, this smart phone becomes First Client 1c and when a doctor logs in, this smart phone becomes Second Client 2c.

The First Client 1c includes the Image Acquiring Module 11c, Voice Inspection Module 12c, Questionnaire Investigation Module 13c, Pulse Diagnosis Module 14c, First Network Communication Module 15c, and First Communication Module 16c. The First Network Communication Module 15c is electrically connected to the Image Acquiring Module 11c, Voice Inspection Module 12c, Questionnaire Investigation Module 13c, Pulse Diagnosis Module 14c, and the First Communication Module 16c.

Image Acquiring Module 11c acquires images from a subject for traditional Chinese medical science health consultation. In this embodiment, this module takes photos of a subject's face and tongue through the smart phone camera.

Voice Inspection Module 12c acquires a subject's voice for traditional Chinese medical science health consultation. For example in this embodiment, the voice inspection module collects a subject's voice, e.g. reading one paragraph or a cough etc.

Questionnaire Investigation Module 13c acquires questionnaire information for a traditional Chinese medical science health consultation. Its purpose is to get a subject's health status through questionnaire.

Pulse Diagnosis Module 14c acquires a subject's pulse information for a traditional Chinese medical science health consultation. See reference FIG. 5, Pulse Diagnosis Module 14c which also includes Pulse Image Collecting Unit 10, Pulse Image Preprocessing Unit 20, and Pulse Condition Extraction Unit 30. Among which, output from Pulse Image Collecting Unit 10 is connected to Pulse Image Preprocessing Unit 20. Output from Pulse Image Preprocessing Unit 20 is connected to Pulse Condition Extraction Unit 30. Output from Pulse Condition Extraction Unit 30 is connected to First Network Communication Module 15a.

Pulse Image Collecting Unit 10 is used for collecting a subject's pulse image. For a smart phone, Pulse Image Collecting Unit 10 is a camera component in a cell phone, including a camera, flash etc. The subject uses a camera to take videos of the pulse and can use the flash to ensure better video quality. Usually by collecting 10 seconds of video can meet the requirement.

Pulse Image Preprocessing Unit 20 is used for processing a pulse image that is collected. Further, the Pulse Image Preprocessing Unit 20 includes Image Sampling Unit 200, Image Clipping Unit 202, and Noise Repair Unit 204. The connective relationship among those units are as follows: the output of the Image Sampling Unit 200 is connected to the Image Clipping Unit 202 and the output of the Image Clipping Unit 202 is connected to the Noise Repair Unit 204.

Image Sampling Unit 200 takes samples using fixed frequency from pulse images collected and gets multiple pulse images. Image Clipping Unit 202 finds out the area with highest luminance for each frame of a pulse image, and uses uniform rectangle window to clip. Noise Repairing Unit 204 repairs noise points in a rectangular window after clipping.

Pulse Condition Extraction Unit 30 extracts pulse condition from preprocessed data. Pulse Condition includes an average of three primary colors (R,G,B) for each frame of a pulse image, variation range for red primary color (R) and pulse frequency. In traditional Chinese medical science, a pulse condition refers to an outside symptom from a pulse beat, which includes pulse frequency, pulse rhythm, pulse position, pulse veins, pulse figure, pulse energy and pulse width. The variation of a pulse condition is closely related to viscera's qi and blood function and status, therefore pulse diagnosis can provide reference data for a traditional Chinese medical science health consultation.

Pulse Condition Extraction Unit 30 also includes Three Primary Colors Average Compute Unit 300, Red Primary Color Variation Range Compute Unit 302 and Pulse Frequency Compute Unit 304.

Three Primary Colors Average Compute Unit 300 computes an average value for each pulse image's three primary colors (R, G, B)

Red Primary Color Variation Range Compute Unit 302 computes variation range for a red primary color based on the red primary color average value in the three primary color average values for each frame of a pulse image.

Pulse Frequency Compute Unit 304 computes a pulse frequency based on a red primary color (R) variation range in each frame of a pulse image.

First Network Communication Module 15a encapsulates the above image, voice, questionnaire information and pulse condition into transmittable data and sends data to Server 3c.

Server 3c includes Server Interface Module 31c and Storage Module 32c. Server Interface Module 31c converts collected transmittable data into an image, voice, questionnaire and pulse condition, and stores them through Storage Module 32c.

Second Client 2c includes Second Network Communication Module 21c and Health Data Visualization Module 22a. Second Network Communication Module 21c receives image and voice data transmitted from Server 3c, and displays those images and voice through Health Data Visualization Module 22c.

A doctor determines the health consultation result based on visualized data and via Server 3c that gives feedback to the subject's First Client 1c.

This embodiment is different from the first embodiment in that the subject's First Client 1c uses the First Communication Module 16c to interact with the Second Client 2c; the Doctor's Second Client 2c uses the Second Communication Module 23c to interact with the First Client 1c. The interaction improves efficiency in health consultation.

A Fourth Embodiment of the Internet-Based Traditional Chinese Medical Science Health Consultation.

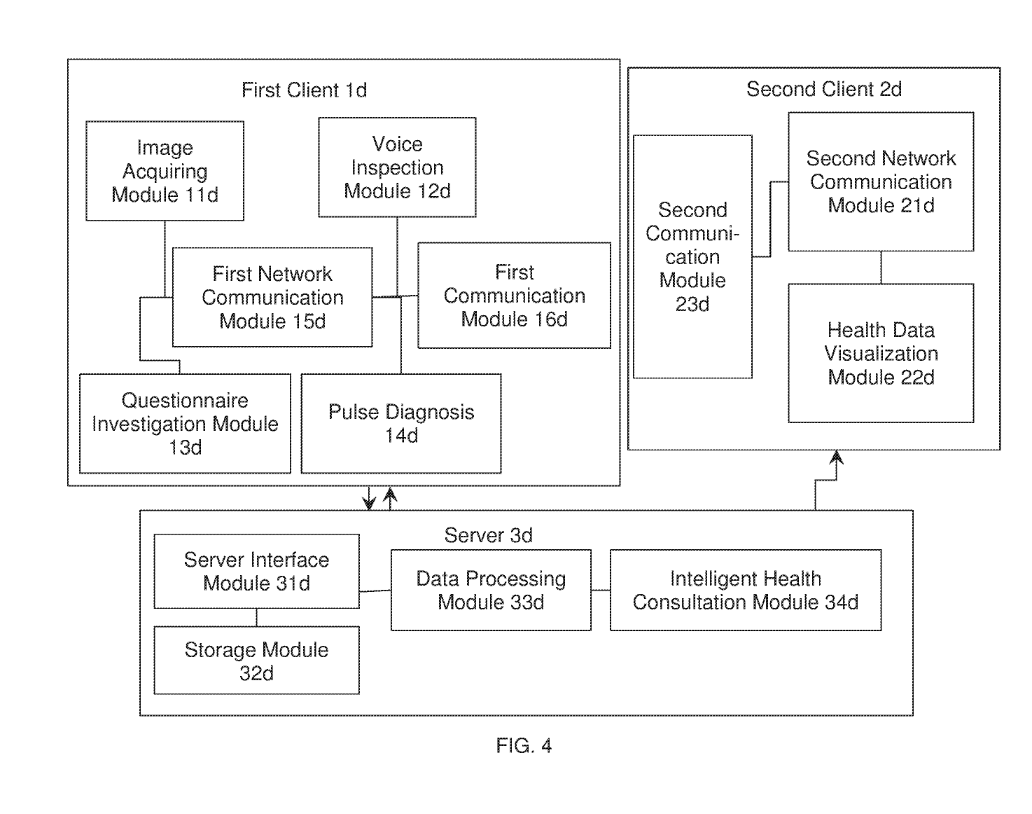

FIG. 4 shows the principle of the fourth embodiment of the internet-based traditional Chinese medical science health consultation in this invention. In reference to FIG. 4, the internet-based traditional Chinese medical science health consultation in this embodiment includes First Client 1d for a subject to login, Second Client 2d for a doctor to login, and Server 3d. Among them, First Client 1d and Second Client 2d are preferably mobile terminals, so they can be an APP installed in a smart phone. When a subject logs in, this smart phone becomes First Client 1d and when a doctor logs in, this smart phone becomes Second Client 2d.

First Client 1d includes Image Acquiring Module 11d, Voice Inspection Module 12d, Questionnaire Investigation Module 13d, Pulse Diagnosis Module 14d, First Network Communication Module 15d, First Communication Module 16d. First Network Communication Module 15d is electrically connected to Image Acquiring Module 11d, Voice Inspection Module 12d, Questionnaire Investigation Module 13d, Pulse Diagnosis Module 14d, and First Communication Module 16d.

Image Acquiring Module 11d acquires images from a subject for traditional Chinese medical science health consultation. In this embodiment, this module takes photos on a subject's face and tongue through a smart phone camera.

Voice Inspection Module 12d acquires voice from a subject for traditional Chinese medical science health consultation. For example in this embodiment, it is to collect the subject's voice, e.g. reading one paragraph or a cough etc.

Questionnaire Investigation Module 13d acquires questionnaire information for a traditional Chinese medical science health consultation. Its purpose is to get a subject's health status through questionnaire.

Pulse Diagnosis Module 14d acquires a subject's pulse information for traditional Chinese medical science health consultation. In reference to FIG. 5, Pulse Diagnosis Module 14d also includes Pulse Image Collecting Unit 10, Pulse Image Preprocessing Unit 20, Pulse Condition Extraction Unit 30. Among which, output from Pulse Image Collecting Unit 10 is connected to Pulse Image Preprocessing Unit 20. Output from Pulse Image Preprocessing Unit 20 is connected to Pulse Condition Extraction Unit 30. Output from Pulse Condition Extraction Unit 30 is connected to First Network Communication Module 15d.

Pulse Image Collecting Unit 10 is used for collecting a subject's pulse image. For a smart phone, Pulse Image Collecting Unit 10 is camera component in a cell phone, including a camera, flash etc. The subject uses a camera to take videos of the pulse and can use the flash to ensure better video quality. Usually collecting 10 seconds' video can meet the requirement.

Pulse Image Preprocessing 20 is used for processing a pulse image that is collected. Further, Pulse Image Preprocessing Unit 20 includes Image Sampling Unit 200, Image Clipping Unit 202 and Noise Repair Unit 204. The relationship of connection among those units are: the output of the Image Sampling Unit 200 is connected to the Image Clipping Unit 202 and the output of the Image Clipping Unit 202 is connected to the Noise Repair Unit 204.

Image Sampling Unit 200 takes samples using fixed frequency from pulse images collected and gets multiple pulse images. Image Clipping Unit 202 finds out the area with highest luminance for each frame of a pulse image, and uses a uniform rectangle window to clip. Noise Repairing Unit 204 repairs noise points in a rectangular window after clipping.

Pulse Condition Extraction Unit 30 extracts a pulse condition from preprocessed data. Pulse Condition includes average of three primary colors (R,G,B) for each frame of a pulse image, variation range for red primary color (R) and pulse frequency. In traditional Chinese medical science, pulse condition refers to outside symptom from pulse beat, which includes pulse frequency, pulse rhythm, pulse position, pulse veins, pulse figure, pulse energy and pulse width. The variation of pulse condition is closely related to viscera's qi and blood function and status, therefore the pulse diagnosis can provide reference data for a traditional Chinese medical science health consultation.

Pulse Condition Extraction Unit 30 also includes Three Primary Colors Average Compute Unit 300, Red Primary Color Variation Range Compute Unit 302 and Pulse Frequency Compute Unit 304.

Three Primary Colors Average Compute Unit 300 computes the average value for each pulse image's three primary color (R, G, B)

Red Primary Color Variation Range Compute Unit 302 computes the variation range for a red primary color based on the red primary color average value in the three primary color average values for each frame of pulse image.

Pulse Frequency Compute Unit 304 computes pulse frequency based on a red primary color (R) variation range in each frame of a pulse image.

First Network Communication Module 15d encapsulates the above image, voice, questionnaire information and a pulse condition into transmittable data and sends the data to Server 3d.

Server 3d includes Server Interface Module 31d, Storage Module 32d, Data Processing Module 33d, Intelligent Health Consultation Module 34d. Server Interface Module 31d converts data received into image, voice, questionnaire information and pulse condition and store them through Storage Module 32d.

Data Processing Module 33d processes received image, voice, questionnaire information and pulse condition in order to provide information for an intelligent health consultation. Data Processing Module 33d includes Image Processing Unit 331d, Voice Processing Unit 332d, Questionnaire Processing Unit 333d, and Pulse Condition Processing Unit 334d.

Image Processing Unit 331d preprocesses an uploaded image from a subject, mainly normalizing luminance and color for face and tongue images.

Voice Processing Unit 332d stores an uploaded voice from the subject (voice includes talking and cough) as a voice file.

Questionnaire Processing Unit 333d stores questionnaire information and scores a questionnaire and stores a score result.

Pulse Condition Processing Unit 334d conducts intelligent pulse diagnosis based on pulse condition using predefined ANN (Artificial Neural Network) model and then achieves pulse diagnosis result. For this embodiment, pulse diagnosis result is the detailed pulse condition, for example Yin Pulse, Yang Pulse, Concurrent Pulse and Flat Pulse etc. In traditional Chinese medical science, pulse condition is firstly classified into Yin and Yang. For example according to the book Typhoid Theory. Pulse Recognizing Method: “If the pulse is big, floating, counting, moving, sliding, it is Yang; if the pulse is falling, obscure, weak, single, tiny, it is Yin.” This means a Yang pulse is a large pulse, with large amplitude, is powerful and fast in pulse frequency. A Yin pulse on the contrary is a shallow pulse. Classification for Yin and Yang pulse and its effect is consistent with Yin and Yang proof in Eight Proofs, i.e. Yin Proof and Yang Proof are the leading proofs in Eight Proofs. The Yin and Yang pulse dominate all pulses, which is the basis for judging sickness exterior/interior, abstract/concrete, cold/hot. Concurrent pulse refers to pulse with both characteristics from Yin and Yang pulse, for example: falling counting pulse, floating slow pulse. Flat pulse refers to the pulse which comes softly, neither fast nor slow, and indicates a healthy pulse condition.

In this embodiment, the Artificial Neural Network model is a four-layer model including three input values (three parameters in pulse condition) and one output value (pulse diagnosis result). The model is generated as follows: use a random parameter to generate it, conduct a supervised learning through knowledge from the expert system module (collected and sorted out in beginning experiment stage), when the model parameter is converging, it indicates a learning step is completed and it forms the Artificial Neural Network model which can be used for health consultation based on pulse condition.

Intelligent Health Consultation Module 34d provides an automatic health consultation result based on output from predefined data analytics model and data processing module 33d and then transmits that to Client 1d through Server Interface Module 31d. This data analytics model is designed based on big data technology and it is achieved by analyzing and studying historical data. Of course, sickness oriented intelligent traditional Chinese medical science health consultation in this embodiment can also be extended to evaluating health status using traditional Chinese medical science.

Second Client 2d includes Second Network Communication Module 21d and Health Data Visualization Module 22d. Second Network Communication Module 21d receives image and voice data transmitted from Server 3d, and displays those images and voice through Health Data Visualization Module 22d.

A doctor determines a health consultation result based on visualized data and via Server 3d which gives feedback to the subject's First Client 1d.

This embodiment is different from first embodiment in that a subject's First Client 1d uses the First Communication Module 16d to interact with the Second Client 2d. The Doctor's Second Client 2d uses the Second Communication Module 23d to interact with the First Client 1d. The interaction improves efficiency in health consultation.

Technicians in this domain will understand further that with the disclosed embodiments in this document, various explanatory logic board, module, circuit and algorithm steps can be implemented as electrical hardware, computer software and combination of the two. To explain clearly the interchange between hardware and software, various explanatory components, frames, modules, circuits and steps mentioned above are described generically with their functions. Whether those functions are implemented as hardware or software depends on concrete application and design constraints on the whole system. Technicians can implement those functions with a different way for each particular application, but such implementation decision should not be explained to be away from this invention's scope.

Based on this document's disclosed embodiments, a various explanatory logical board, module and circuit can be implemented or executed using generic processor, Digital Signal Processor (DSP), Application-specific integrated circuit (ASIC), Field Programmable Gate Array (FPGA) or other programmable logical components, discrete gate or transistor logic, discrete hardware components or any combinations for executing this document's functions. Generic processor can be a micro-processor, but in an alternative solution, this processor can be any normal processor, controller, micro controller or state machine. Processor can be implemented as a combination of a computing device, for example combination of DSP and micro-processor, multiple micro-processors, one or more micro-processors collaborating with DSP core and any other such configurations.

Based on this document's disclosed embodiments, the methods or algorithms can be implemented directly in hardware, or software modules in processor or combination of the two. Software module can stay in RAM storage, flash storage, ROM storage, EPROM storage, EEPROM storage, register, hard disk, mobile disk, CD-ROM, or any other storage media in this domain. Example storage media is coupled into processor so that this processor can read and write information from and to this media. In alternative solution, storage media can be integrated into a processor. Processor and storage media can stay in ASIC. ASIC can stay in user terminal. In an alternative solution, a processor and storage media can stay in user terminal as a discrete component.

In one or multiple example embodiments, the function described can be implemented in hardware, software, firmware or any combination. If they are implemented in software as computer program, then each function can be stored in computer readable media or transmitted through the media as one or more instructions or code. Computer readable media includes computer storage media and communication media, which includes any media which causes a computer program to transmit from one place to another place. Storage media can be any useable media accessible by a computer. As an example but not forced, such computer readable media can include RAM, ROM, EEPROM, CD-ROM or other CD storage, disk storage or other magnetic storage or any other media which can be used to carry or store program code accessible by a computer with the format of instruction or data structure. Any connection can also be appropriately called computer readable media. For example, if software is transmitted through a coaxial cable, optical cable, twisted pair, Digital Subscriber Line (DSL) or wireless technology for example: infrared ray, radio and microwave or other remote source, such technology is included as media. For example, this document uses disk and disc including compress disk (CD), laser disk, optical disk, digital versatile disk, floppy disk and Blu-ray disk, among which disk represents data via magnetic, while disc represents data via optical. A combination of the above should also be included in the scope of computer readable media.

Throughout the description and drawings, example embodiments are given with reference to specific configurations. It will be appreciated by those of ordinary skill in the art that the present invention can be embodied in other specific forms. Those of ordinary skill in the art would be able to practice such other embodiments without undue experimentation. The scope of the present invention, for the purpose of the present patent document, is not limited merely to the specific example embodiments or alternatives of the foregoing description.

What is claimed is:

1. An internet-based traditional Chinese medicine science health consultation system comprising:
a first client, used for a subject to login for a traditional Chinese medical science health consultation, the first client including
an image acquiring module which acquires an image of the subject for the traditional Chinese medical science health consultation,
a voice inspection module which acquires a voice of the subject for the traditional Chinese medical science health consultation,
a questionnaire investigation module which acquires a questionnaire information of the subject for the traditional Chinese medical science health consultation,
a pulse diagnosis module which acquires a pulse condition information of the subject for the traditional Chinese medical science health consultation, and
a first network communication module;
a second client, used for a doctor to login, the second client including a second network communication module and a health data visualization module; and
a server including a server interface module that converts data from the first client and the second client to image, voice, questionnaire, and pulse condition information; and a storage module which stores data from the first client and second client;
wherein the first network communication module is connected to the image acquiring module, the voice inspection module, the questionnaire investigation module, and the pulse diagnosis module, respectively, and encapsulates the image, voice, questionnaire information, and pulse condition information (hereinafter referred to as “the information”) and then transmits the information to the server;
wherein the second network communication module is used to communicate with the first client and the server, the second network communication module receives data collected from the first client and sends health consultation feedback from the doctor to the first client; and
wherein the health data visualization module is used for receiving image and voice information transmitted from the server and displaying said image and voice information to the doctor.

2. The system according to claim 1, further comprising:
a data processing module which processes the information based on which intelligent health consultation is conducted; and
an intelligent health consultation module, based on a predefined data analytics model and output from the data processing module, provides an automatic health consultation result, and then transmits the result to the first client via the server interface module.

3. The system according to claim 1, wherein the first network communication module is used for communicating with the second client, and the second network communication module is used for communicating with the first client.

4. The system according to claim 2, wherein the data processing module further comprises:
an image processing unit, which performs preprocessing of an image which the subject uploaded and normalizes luminance and color of the image;
a voice processing unit, which stores voice which the subject uploaded to a sound file;
a questionnaire information processing unit, which stores the questionnaire, scores the questionnaire, and stores a score result; and
a pulse information processing unit, based on a predefined ANN (Artificial Neural Network) model, which processes pulse information, performs intelligent health consultation, and provides a pulse diagnosis result.

5. The system according to claim 1, wherein the pulse diagnosis module further comprises:
a pulse image collecting unit, which collects a pulse image from the subject;
a pulse image preprocessing unit, connected to the pulse image collecting unit, the pulse image preprocessing unit performs preprocessing of the pulse image collected from the subject; and
a pulse condition extraction unit, connected to the pulse image collecting unit, the pulse condition extraction unit extracts pulse condition information from data after preprocessing.

6. The system according to claim 5, wherein the pulse image preprocessing module further comprises:
an image sampling unit, which extracts samples out of the pulse image in a fixed frequency and obtains multiple frames of the pulse image
an image clipping unit, connected to the image sampling unit, the image clipping unit detects the area with highest luminance for each frame of the pulse image and uses a uniform rectangle window to clip; and
a noise repairing unit, connected to the image clipping unit, the noise repairing unit repairs noise points in the window after clipping.

7. The system according to claim 5, wherein the pulse condition extraction module further comprises:
a three primary colors average computing unit, which calculates an average value of three primary colors for each frame of the pulse image;
a red primary color variation range computing unit, based on an average red primary color in average three primary colors for each frame of the pulse image, calculates a variation range for red primary color; and
a pulse frequency computing unit, based on a red primary color variation range, for each frame of the pulse image, which calculates a pulse frequency.

8. The system according to claim 7, wherein the pulse condition information includes the average value of three primary colors for each frame of the pulse image, the variation range for red primary color, and the pulse frequency.

9. The system according to claim 1, wherein the image acquiring module acquires the subject's face image and tongue image.

10. The system according to claim 1, wherein the first client and the second client are a mobile terminal.

11. An internet-based traditional Chinese medicine science health consultation system comprising:
a client, used for a subject to login for a traditional Chinese medical science health consultation, the client including
an image acquiring module which acquires an image of the subject for the traditional Chinese medical science health consultation,
a voice inspection module which acquires a voice information of the subject for the traditional Chinese medical science health consultation,
a questionnaire investigation module which acquires a questionnaire information of the subject for the traditional Chinese medical science health consultation,
a pulse diagnosis module which acquires a pulse condition information of the subject for the traditional Chinese medical science health consultation, and
a network communication module; and
a server including
a server interface module which converts data from the client to image, voice, questionnaire, and pulse condition information;
a storage module which stores data from the client;
a data processing module which processes the image, voice, questionnaire, and pulse condition information based on which intelligent health consultation is conducted; and
an intelligent health consultation module, based on a predefined data analytics model and an output from the data processing module, which provides an automatic health consultation result and then transmits the result to the client via the server interface module;
wherein the network communication module is connected to the image acquiring module, the voice inspection module, the questionnaire investigation module, and the pulse diagnosis module respectively, and encapsulates the image, voice, questionnaire, and pulse condition information (hereinafter referred to as “the information”) and then transmits the information to the server.

12. The system according to claim 11, wherein the data processing module further comprises:
an image processing unit, which performs preprocessing of an image which the subject uploaded and normalizes luminance and color of the image;
a voice processing unit, which stores voice which the subject uploaded to a sound file;
a questionnaire information processing unit, which stores the questionnaire, scores the questionnaire, and stores a score result; and
a pulse information processing unit, based on a predefined ANN (Artificial Neural Network) model, which processes pulse information, performs intelligent health consultation, and provides a pulse diagnosis result.

13. The system according to claim 11, wherein the pulse diagnosis module further comprises:
a pulse image collecting unit, which collects a pulse image from the subject;
a pulse image preprocessing unit, connected to the pulse image collecting unit, the pulse image preprocessing unit performs preprocessing of the pulse image collected from the subject; and
a pulse condition extraction unit, connected to the pulse image collecting unit, the pulse condition extraction unit extracts pulse condition information from data after preprocessing.

14. The system according to claim 11, wherein the pulse image preprocessing module further comprises:
an image sampling unit, which extracts samples out of the pulse image in a fixed frequency and obtains multiple frames of the pulse image
an image clipping unit, connected to the image sampling unit, the image clipping unit detects the area with highest luminance for each frame of the pulse image and uses a uniform rectangle window to clip; and
a noise repairing unit, connected to the image clipping unit, the noise repairing unit repairs noise points in the window after clipping.

15. The system according to claim 13, wherein the pulse condition extraction module further comprises:
a three primary colors average computing unit, which calculates an average value of three primary colors for each frame of the pulse image;
a red primary color variation range computing unit, based on an average red primary color in average three primary colors for each frame of the pulse image, calculates a variation range for red primary color; and
a pulse frequency computing unit, based on a red primary color variation range, for each frame of the pulse image, which calculates a pulse frequency.

16. The system according to claim 15, wherein the pulse condition information includes the average value of three primary colors for each frame of the pulse image, the variation range for red primary color, and the pulse frequency.

17. The system according to claim 11, wherein the image acquiring module acquires the subject's face image and tongue image.
